(19) United States
(12) Patent Application Publication
(10) Pub. No.: US 2004/0106588 A1
(43) Pub. Date: Jun. 3, 2004

(54) Topical application of alpha-DFMO and steroid to treat Vulvar Intraepithelial Neoplasia

(76) Inventors: David S. Alberts, Tucson, AZ (US); Robert T. Dorr, Tucson, AZ (US)

Correspondence Address: CAHILL, von HELLENS & GLAZER P.L.C., 155 Park One, 2141 East Highland Avenue, Phoenix, AZ, 85016, US

(21) Appl. No.: 10/655,085

(22) Filed: Sep. 3, 2003

Related U.S. Application Data
(63) Continuation-in-part of application No. 10/041,236, filed on Jan. 7, 2002, now abandoned.

(51) Int. Cl. A61K 31/573

(52) U.S. Cl. 514/179; 514/708

(57) ABSTRACT

A method for treating pre-cancerous conditions including non-cancerous lesions and non-invasive carcinoma insitu, in cutaneous, and muco-cutaneous regions of the body includes topical application of combinations containing alpha-DFMO and an anti-inflammatory agent selected from the group comprising a steroidal anti-inflammatory agent, a non-steroidal anti-inflammatory agent, or a combination thereof. The conditions which may be topically treated with the combination of the present invention include actinic keratoses, vulvar neoplasia, and pre-cancerous conditions of the lip, nostrils, nails, and anus. In one case, the topical steroid triamcinolone is combined with the alpha-DFMO. In a second case, the topical non-steroid anti-inflammatory diclofenac is combined with the alpha-DFMO. In a third instance, both triamcinolone and diclofenac are combined with the alpha-DFMO.

### CROSS-REFERENCE TO RELATED APPLICATIONS

This patent application is a continuation-in-part patent application based upon co-pending U.S. patent application Ser. No. 10/041,236, filed Jan. 7, 2002, and the benefit of such earlier filing date is hereby claimed by Applicant under 35 U.S.C.

### STATEMENT REGARDING FEDERALLY-SPONSORED RESEARCH

The invention was made with government support under Grant No. CA-27502 awarded by the National Institutes of Health (NIH) of Bethesda, Md. to University of Arizona. The U.S. Government has a paid-up license in this invention.

### BACKGROUND OF THE INVENTION

1. Field of the Invention

The present invention relates generally to the fields of cancer biology and biochemistry. More particularly, the invention is directed to a method of treatment of pre-cancerous conditions in cutaneous and muco-cutaneous regions of a human being.

2. Description of the Relevant Art

It is well-documented that squamous cell carcinoma and basal cell carcinoma typically develop within or adjacent to areas of pre-existing pre-malignant actinic keratoses (AKs). The presence of AKs represents a major risk factor for skin cancers. There is strong evidence that the incidence of skin cancer is increasing throughout the United States and other countries, particularly in regions closer to the equator where sunlight is more intense. Incidence rates for skin cancer are expected to increase further as the population ages and larger amounts of UV radiation reach the surface of the earth. Although the mortality rate for these skin cancers is low, their treatment is associated with considerable morbidity and remarkably high medical costs.

Vulvar Intraepithelial Neoplasia (VIN), which is an HPV associated neoplasia, is a pre-cancerous condition which may lead to the occurrence of invasive squamous cell cancer. Though the disease is very stable with a long pre-cancerous phase before invasion, it is generally very resistant to treatment. VIN is a pre-cancerous condition which may be surgically treated to prevent the occurrence of invasive squamous cell cancer. Recurrences following surgical treatment are the rule rather than the exception. The incidence of VIN is increasing and affects younger women. Although screening programs have not been utilized in this pre-cancerous process, it is the general impression among clinicians in this field that the incidence is on the rise. The rationale for this impression may be secondary to the increasing incidence of HPV-related conditions in the female genital tract. Grade III VIN, or VIN III, is likely to develop into cancer from about 70 to 90% of the time.

Treatment of pre-cancerous conditions occurring in cutaneous and muco-cutaneous regions of the body, such as the skin or vulva, is often accompanied by irritation and other ill-tolerated side affects, as these regions are especially sensitive. The most common treatment of AKs continues to be the topical application of 5% fluorouracil cream, or liquid nitrogen. Both of these methods result in severe inflammation, erythema, and superficial ulceration. There continues to be a need for the development of less toxic drugs which can be applied chronically as chemopreventive agents for patients with severely sun-damaged skin and AKs.

Traditional treatments for VIN, and particularly VIN III related lesions, include surgical excision or vaporization with a CO₂ laser. However, due to an overall field effect for HPV-related change in the vulva, recurrence is common, often multifocal, and repetitive surgeries can often lead to significant scarring and discomfort. Even the use of skin grafting does not preclude recurrence, as there have been reports of recurrences in the grafts. Topical application of 5-fluorouracil (5-FU)-4 has also been utilized as an alternative treatment. However, 5-FU, though relatively well tolerated in the vagina, is extremely poorly tolerated on the vulva. Patients experience intense pain and burning, and compliance is poor.

Alpha-Difluoromethylornithine (DFMO), chemical formula C₆H₁₅CIF₂N₂O₃, is an enzyme-activated irreversible inhibitor of ornithine decarboxylase, which is the rate-limiting enzyme in polyamine synthesis, and decreases intracellular levels of putrescine and spermidine in the skin and other vital tissues. In conjunction with the administration of model carcinogens, DFMO has been shown to significantly reduce tumor incidence in several mammalian in vivo tests for chemopreventive activity.

The major effort to date in studying DFMO has been in colon cancer. Reddy et al., “Chemoprevention of colon carcinogenesis by concurrent administration of piroxicam, a non-steroidal anti-inflammatory drug with D, L-alpha difluoromethylomithine, an ornithine decarboxylase inhibitor, in diet, ” Cancer Res 50(9):2562-8, 1990, refer to studies demonstrating that NSAIDs (Non-Steroidal Anti-Inflammatory Drugs) such as piroxicam, ibuprofen, aspirin and sulindac, D, L-alpha-difluoromethylomithine, oltipraz, anethole trithione, and diallyl disulfide inhibited colon adenocarcinomas. This article also reports results of a study involving Piroxicam and DFMO, which indicated that the combination of piroxicam and DFMO had a stronger inhibitory effect than when the two drugs were administered individually.

Zirvi and Atabek, “In vitro response of a human colon tumor xenograft and a lung adenocarcinoma cell line to alpha-difluoromethylornithine alone and in combination with 5-fluorouracil and doxorubicin, ” J Surg Oncol 48(1):34-8, 1991, investigated the effects of alpha DFMO on human colon tumor xenograft (T6) cells and a human lung adenocarcinoma cell line (A549), reporting that DFMO showed a dose-dependent growth-inhibitory effect. Meyskens and Gerner, “Development of difluoromethylomithine as a Chemoprevention agent for the management of colon cancer, ” J Cell Biochem Suppl 22:126-31, 1995, conducted a Phase II trial to determine the lowest dose at which DFMO decreased colon mucosa polyamine content, finding that an oral dose as low as 0.25 gm/m² per day decreased colon tissue putrescence content and lowered the spermidine/spermine ratio.

Loser et al, “Dissimilar activation patterns of the carcinogen dimethylhydrazine (DMH) on intracellular polyamine metabolism in various organs,” Z Gastroenterol 34(12):801-8, 1996, reported that DFMO treatment completely prevented DMH-induced activation of polyamine de novo synthesis and DNA polymerase in the colon and the small intestine.

DFMO has been also studied in a phase I format in cervical intraepithelial (CIN) neoplasia, wherein a trial with 30 women having biopsy-proven cervical intraepithelial neoplasia level III was conducted. (see Mitchell et al, Results of Phase I Chemoprevention Trial of Alpha-Difluoromethylomithine (DFMO) in Patients with Cervical Intraepithelial Neoplasia Grade 3, Gyn Oncology 60(1):101, 1996). Doses of DFMO ranged from 0.06 g/m² per day up to 1.0 g/m² per day for a total of 30 days. Patients then underwent a loop excision of the cervical transformation zone. An overall 50% response rate was seen, with five out of thirty patients experiencing a complete response and ten out of thirty experiencing a partial response, or a reduction to levels I or II despite the short treatment interval. The study further reported minimal toxicity, and significant tissue polyamine modulation down to a dose level of 0.5 g/m² per day, while serum polyamine modulation was significant down to a dose of 0.125 g/m² per day.

Also, Zou, et al, “Effects of Difluoromethylomithine on Growth Inhibition and Apoptosis in Human Cervical Epithelial and Cancerous Cell Lines,” Gyn Oncol 85, 266-273, 2002, reported that DFMO inhibits the growth of cervical pre-cancerous and cancerous cells in vitro in a dose dependent and time dependent manner, partially through inducing apoptosis.

However, a limiting side effect accompanying oral use of DFMO is a reversible ototoxicity, resulting in problems such as hearing loss. In fact, DFMO has been utilized in multiple phase I trials to determine appropriate dosing. Meyskens et al, “Development of difluoromethylomithine as a chemoprevention agent for the management of colon cancer” J Cell Biochem Suppl 22:126-31, 1995, carried out phase I trials to determine the dose at which ototoxicity was not relevant yet endpoint biomarkers demonstrated significant activity, reporting that this was achieved at a dose of 250 mg/m² per day.

DFMO chemopreventive activity has also been demonstrated in chemical and UV models of mouse skin carcinogenesis. It has also been shown that p.o.-administered DFMO reduced UVB-induced skin cancers in C3H/HeN mice from 38% in placebo treated controls to 9% in treated animals. Similarly, topically administered DFMO in an acetone vehicle dramatically reduced UVB-induced skin cancers in BALB/c mice. In adult participants with psoriasis, the application of 10% DFMO cream resulted in a 66% reduction in spermidine concentrations in the skin and a marginal improvement in psoriatic lesions.

Gerner, U.S. Pat. No. 6,258,845, discloses the use of DFMO and NSAID for treatment of various cancers, including skin and cervical cancer. Gerner also mentions various possible transmission routes which include topical and vaginal, but does not mention use of DFMO for treating vulvar neoplasia, nor the combination of DFMO with steroids.

Also, within U.S. Pat. No. 5,851,537, applicants disclosed the topical application of alpha-DFMO in a hydrophilic cream salve carrier for use as a topical chemopreventive agent against skin cancer. As set forth in such patent, tests on human subjects showed that the topical application of such cream reduced the number of AKs on the arms of such subjects. However, several of the subjects experienced skin rashes in the area where the alpha-DFMO was applied.

Severe skin irritation has also been noted as a negative side effect with topical use of fluorouracil for treating actinic keratoses or vulvar neoplasia. Back in 1976, Breza, et al., “Noninflammatory Destruction of Actinic Keratoses by Flourouracil”, Arch Dermatol, Vol. 112, September 1976, pp. 1256, proposed that 0.5% triamcinolone acetonide cream be added to topical compositions of fluorouracil in order to suppress the inflammatory reaction associated with the topical fluorouracil therapy of actinic keratoses. Breza, et al. concluded that the addition of 0.5% triamcinolone acetonide did not alter the effectiveness of the fluorouracil, but that it did serve to reduce inflammatory response.

Diclofenac is a topical NSAID (non-steroidal anti-inflammatory drug), and has been used in the past for the treatment of pain and inflammation in rheumatoid arthritis by inhibiting cyclooxygenase enzymes. More recently, the FDA has approved the drug diclofenac in topical gel form, available from Skylabs/SkyePharma Inc. of San Diego, Calif. and/or its British affiliate, under the trade designation “Solareze”, for treatment of actinic keratoses. It belongs to the family of medicines called antineoplastics, at least some of which are believed to kill cancerous cells.

While some of the compositions incorporating DFMO mentioned above have been shown to have varying degrees of effectiveness in treating actinic keratoses, there continues to be a need for more effective and less irritating treatments. Additionally, although oral use of DFMO has also been shown to have some degree of effectiveness for treating neoplasia in the cervix, applicants are not aware of its use for treating neoplasia in the vulva, which, unlike the cervix, is a muco-cutaneous member, and can not be removed, as can the cervix.

In view of the foregoing, it is an object of the present invention to provide a topical composition and method for treating pre-cancerous conditions of cutaneous and muco-cutaneous regions which is effective and well tolerated.

It is also an object of the present invention to provide such a topical composition and method that is less likely to be toxic.

Yet another object of the present invention is to enhance the effectiveness of alpha-DFMO when treating actinic keratoses and vulvar neoplasia in humans.

It is a further object of the present invention to provide a topical composition and method for treating actinic keratoses and vulvar neoplasia using reduced concentrations of alpha-DFMO without compromising the effectiveness of the treatment.

Still another object of the present invention is to enhance the effectiveness of the drug diclofenac when topically treating pre-cancerous conditions of cutaneous and muco-cutaneous regions in humans.

These and other objects of the present invention will become more apparent to those skilled in the art as the description of the present invention proceeds.

### SUMMARY OF THE INVENTION

Briefly described, and in accordance with one embodiment of the thereof, the present invention relates to a method for treating pre-cancerous conditions including non-cancerous lesions and non-invasive carcinoma insitu, in cutaneous, and muco-cutaneous regions of the body by topical application of combinations containing alpha-DFMO and an anti-inflammatory agent selected from the group comprising a steroidal anti-inflammatory agent, a non-steroidal anti-inflammatory agent, or a combination thereof. The conditions which may be topically treated with the combination of the present invention include actinic keratoses, vulvar neoplasia, and pre-cancerous conditions of the lip, nostrils, nails, and anus.

In practicing such method, a topical steroid, preferably topical triamcinolone (a corticosteroid; chemical formula C₂₄H₃₁FO₆), is applied in conjunction with the alpha-DFMO composition. According to a preferred embodiment, alpha-DFMO and triamcinolone are combined into a single formulation and applied to the affected region. Alternatively, a first formulation containing alpha-DFMO and a second formulation containing triamcinolone are each applied at least once at different times during a 24 hour period. If desired, the first and second formulations may be stored separately, then mixed together prior to application. Preferably the alpha-DFMO and triamcinolone are separately, or collectively, combined with a base vehicle such as a hydrophilic ointment (Hydrophilic Ointment, USP), Vanicream® topical vanishing-cream, or the like; such base vehicles can include both water-in-oil emulsions and oil-in-water emulsions.

Though testing is yet to be conducted, applicants believe that other topical steroidals may be substituted for topical triamcinolone, including betamethasone, clobetasol, dexamethasone, furoate, fluocinonide, amcinonide, desonide, desoximetasone, fluocinolone, fluticasone, halobetasol, hydrocortisone, and mometasone.

This combination is applied topically to the cutaneous or muco-cutaneous regions of a human being having pre-cancerous conditions in those regions. This includes applying the combination to the vulvar region of a human being having VIN and VIN associated lesions, which are well pronounced in VIN III, including primary or recurrent, unifocal or multifocal lesions, in order to reduce the number of such lesions, and to reduce associated spermidine concentrations. The combination may also be used to treat lower grades of VIN, including VIN I where lesions may not be visible if at all present. Likewise, the combination is applied topically to the skin of a human being having actinic keratoses lesions in order to reduce the number of such actinic keratoses lesions and to reduce spermidine concentrations associated with such skin tissues. Furthermore, the combination may be applied to treat pre-cancerous conditions of the lip, nails, nostrils, and anus.

Preferably, topical alpha-DFMO represents from 0.1% to 20% by weight of the applied combination. It is also preferred that topical triamcinolone be present within the range of 0.001% to 1.0% by weight. Preferred relative ratios by weight of alpha-DFMO to triamcinolone combined with the base vehicle ranges between 0.1:2 and 40:1.

In accordance with another embodiment thereof, the present invention relates to a method for treating pre-cancerous conditions in cutaneous and muco-cutaneous regions, including actinic keratoses, vulvar neoplasia, and pre-cancers of the lip, nails, nostrils, and anus, by topical application to the cutaneous or muco-cutaneous tissues of a human being a combination comprising alpha-DFMO and a topical non-steroid anti-inflammatory, preferably diclofenac. The non-steroid anti-inflammatory and DFMO may be separately applied at different times, or may be applied together in a single formulation. Also, the alpha-DFMO and diclofenac may be separately formulated and stored, then mixed together prior to application. The DMFO and anti-inflammatory are preferably added to a base vehicle, such as a hydrophilic ointment (Hydrophilic Ointment, USP), Vanicream® topical vanishing cream or the like, separately or together. This combination is applied topically to the cutaneous or muco-cutaneous regions of a human being having pre-cancerous conditions in those regions. This includes applying the combination to the vulvar region of a human being having VIN and VIN associated lesions, which are well pronounced in VIN III, including primary or recurrent, unifocal or multifocal lesions, in order to reduce the number of such lesions, and to reduce associated spermidine concentrations. The combination may also be used to treat lower grades of VIN, including VIN I where lesions may not be visible if at all present. Likewise, the combination is applied topically to the skin of a human being having actinic keratoses lesions in order to reduce the number of such actinic keratoses lesions and to reduce spermidine concentrations associated with such skin tissues. Furthermore, the combination may be applied to treat pre-cancer occurring in the lip, nails, nostrils, and anus.

Other topical non-steroid anti-inflammatory drugs which may be used in place of diclofenac include difunisal, etodolac, fenoprofen, ketoprofen, ketorolac, mefenamic acid, nabumetone, naproxen, oxaprozin, tolmetin sodium, ibuprofen, celecoxib, rofecoxib, choline salicylate, and sodium salicylate. Preferably, the alpha-DFMO is present within the range of 0.1% to 20% by weight; likewise, in the preferred embodiment, the topical diclofenac has a concentration within the range of 0.1%-10% by weight. The relative ratio of alpha-DFMO to topical diclofenac, by weight, within the preferred embodiment, lies between 1:10 and 200:1, whether in a single combined formulation or two separate formulations.

The present invention also relates, in conjunction with another embodiment, to a method for treating pre-cancerous conditions in cutaneous and muco-cutaneous regions, including actinic keratoses and vulvar neoplasia, and pre-cancers of the lip, nails, nostrils, and anus, by topical application of a three-drug combination comprising alpha-DFMO, a topical steroid, preferably triamcinolone, and a topical non-steroid anti-inflammatory, preferably diclofenac. This combination may be formulated in one base vehicle. Alternatively, each of the three ingredients may be combined with separate base vehicles, wherein each of the three formulations is applied separately. As another alternative, two of the ingredients may be combined in one formulation, with the third ingredient carried by a separate base vehicle and both would be applied separately within a twenty-four hour period. The different formulations may also be stored separately, but mixed with each other prior to application. The combination is applied topically to the cutaneous or muco-cutaneous regions of a human being having pre-cancerous conditions in those regions. This includes applying the combination to the vulvar region of a human being having VIN and VIN associated lesions, which are well pronounced in VIN III, including primary or recurrent, unifocal or multifocal lesions, in order to reduce the number of such lesions, and to reduce associated spermidine concentrations. The combination may also be used to treat lower grades of VIN, including VIN I where lesions may not be visible, if at all present. Likewise, the combination is applied topically to the skin of a human being having actinic keratoses lesions in order to reduce the number of such actinic keratoses lesions and to reduce spermidine concentrations associated with such skin tissues. Furthermore, the combination may be topically applied to the lip, nails, nostrils, and anus to treat pre-cancerous conditions of those regions. Again, the base vehicle is preferably a hydrophilic ointment (Hydrophilic Ointment, USP), Vanicream® topical vanishing cream, a water-in-oil emulsion, an oil-in-water emulsion, or the like.

### BRIEF DESCRIPTION OF THE DRAWINGS

FIG. 1 is a graph illustrating the comparative impact of using alpha-DFMO alone, versus alpha-DFMO plus triamcinolone, in reducing the survival rate of I-7 human ras-transfected epidermal keratinocytes.

FIG. 2 is a table setting forth test data showing the relative effects of using alpha-DFMO alone, triamcinolone alone, and combinations of alpha-DFMO plus triamcinolone, in varying combinations, in order to reduce the survival rate of I-7 human ras-transfected epidermal keratinocytes.

FIG. 3 is a graph illustrating the comparative impact of using alpha-DFMO alone, versus alpha-DFMO plus triamcinolone, in reducing the survival rate of 11-4 human squamous skin cancer cells.

FIG. 4. is a table setting forth test data showing the relative effects of using alpha-DFMO alone, triamcinolone alone, and combinations of alpha-DFMO plus triamcinolone, in varying combinations, in order to reduce the survival rate of 11-4 human squamous skin cancer cells.

FIG. 5 is a graph illustrating the combined effect of topical alpha-DFMO and triamcinolone on human II-4 squamous skin cancer cells injected into SCID mice.

FIG. 6 is a graph illustrating the impact of alpha-DFMO and triamcinolone, alone and in combination, upon growth rates of human squamous cell skin cancer volume.

FIG. 7 is a data table showing the results of an in vitro evaluation of the topical combination of alpha-DFMO, triamcinolone, and diclofenac upon the growth of II-4 human squamous skin cancer cells.

### DETAILED DESCRIPTION OF THE PREFERRED EMBODIMENTS

As mentioned above, alpha-DFMO is an enzyme-activated irreversible inhibitor of omithine decarboxylase, and it acts to decrease intracellular levels of putrescine and spermidine in the skin. For purposes of the practice of the present invention, alpha-DFMO was formulated by starting with a white powder of the monohydrate, monochloride form, having a molecular weight of 236.65, commercially available originally from Marrion-Merrell Dow Pharmaceutical Company of Kansas City, Mo., and now available from Sigma Chemicals, St. Louis, Mo. for in vitro and in vivo mouse studies, and from ILEX Oncology, San Antonio, Tex. for clinical studies. Such alpha-DFMO is mixed with a base vehicle for topical application. The preferred base vehicle is a hydrophilic ointment (Hydrophilic Ointment, USP) commercially available from E. Fougera & Company of Melville, N.Y. Preferably, the alpha-DFMO comprises 10% by weight of the formulation.

For purposes of practicing the present invention, triamcinolone (triamcinolone acetonide) was of the type formulated by E. Fougera & Company of Melville, N.Y. Appropriate amounts of the alpha-DFMO and/or triamcinolone were weighed-out and mixed in a blender with the hydrophilic ointment. Preferably, the triamcinolone comprises 0.1% by weight of the formulation. Once mixed, the combination was transferred to polyethylene-lined, 30 gram metal ointment tubes which were crimp-sealed to preclude exposure to light and air. In some studies, control groups received pure hydrophilic ointment applied topically, or no treatment at all.

It was found that the addition of the topical steroid reduces alpha-DFMO induced inflammatory response in the skin. As mentioned above, topical steroids have been used in the past to reduce skin inflammation. Surprisingly, however, the in vitro addition of the topical steroid under cell culture conditions has been found to significantly enhance the effectiveness of alpha-DFMO in reducing the growth of immortalized human keratinocytes and ras-transfected human squamous cell keratinocytes. Additionally, the addition of the topical steroid has been found to significantly enhance the effectiveness of topical alpha-DFMO in reducing squamous cell skin tumors implanted in immunodeficient mice. In other words, the combination of the topical steroid triamcinolone with topical alpha-DFMO has shown an unpredictable synergistic effect relative to reduction of squamous cell skin tumors.

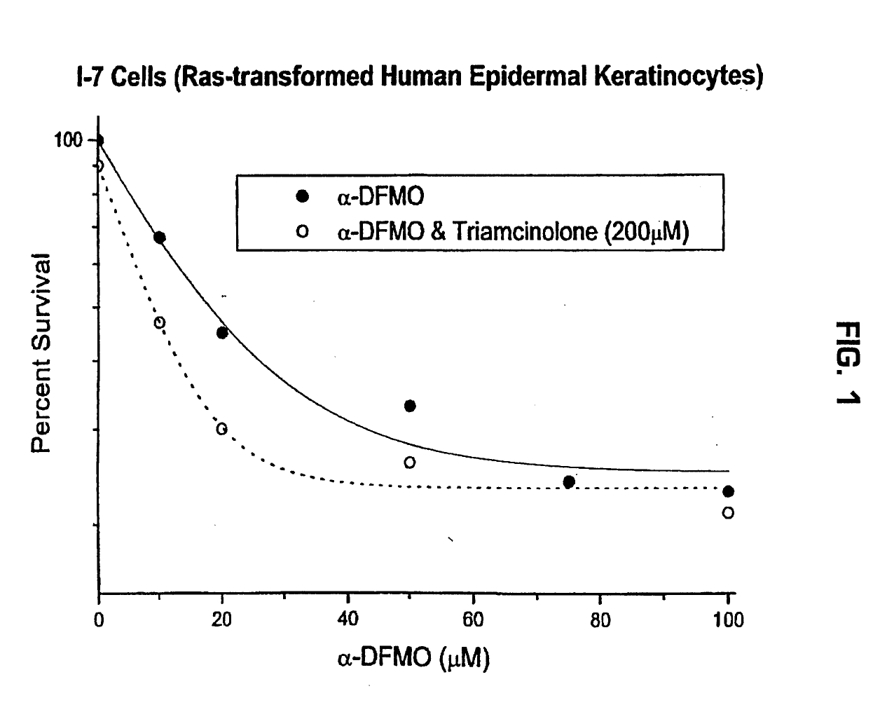

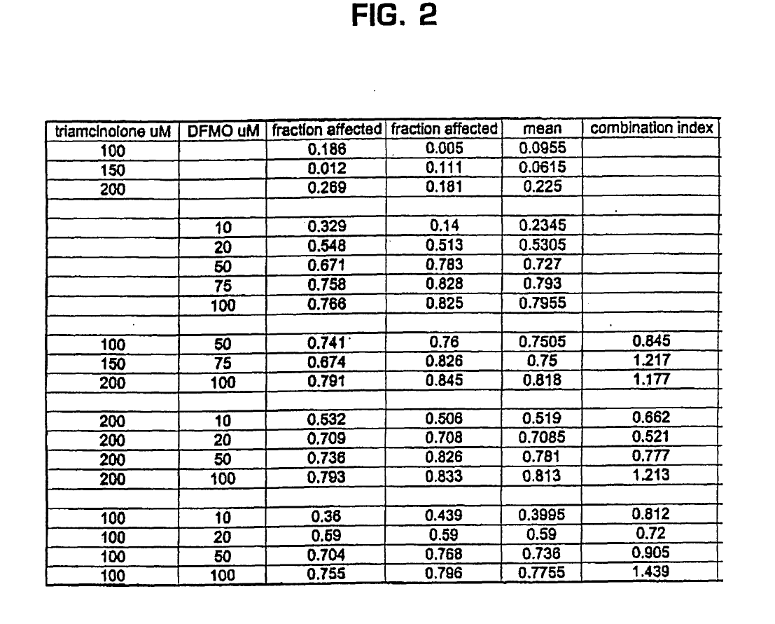

One method used to demonstrate the unexpected effectiveness of the combination of the topical steroid triamcinolone with topical alpha-DFMO was an in-vitro study performed in a laboratory using two different human cutaneous cell lines transfected with a mutated ras gene. The Median Dose Effect Principal Method was used to evaluate additivity and synergism of the two components alpha-DFMO triamcinolone and triamcinolone. The graph set forth in FIG. 1 shows the percent survival rate of Type I-7 transformed keratinocyte cells as a function of the microMolar concentration of the alpha-DFMO. The FIG. 1 graph demonstrates that, for concentrations of alpha-DFMO in the range of approximately 10 to 60 microMolar, the addition of 200 microMolar triamcinolone significantly improved the effectiveness of alpha-DFMO alone relative to the reduction of Type I-7 actinic keratoses cells. All of the data recorded for Type I-7 actinic keratoses cells is set forth in the data table of FIG. 2.

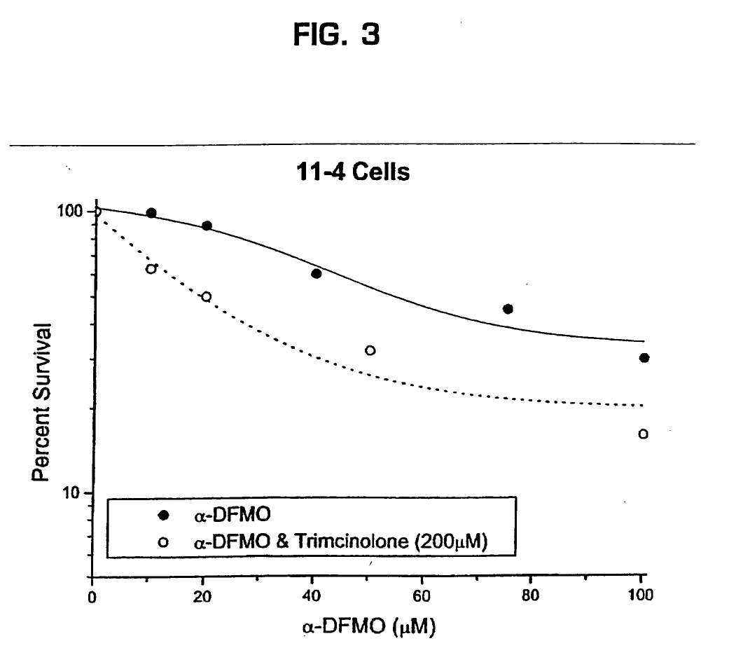

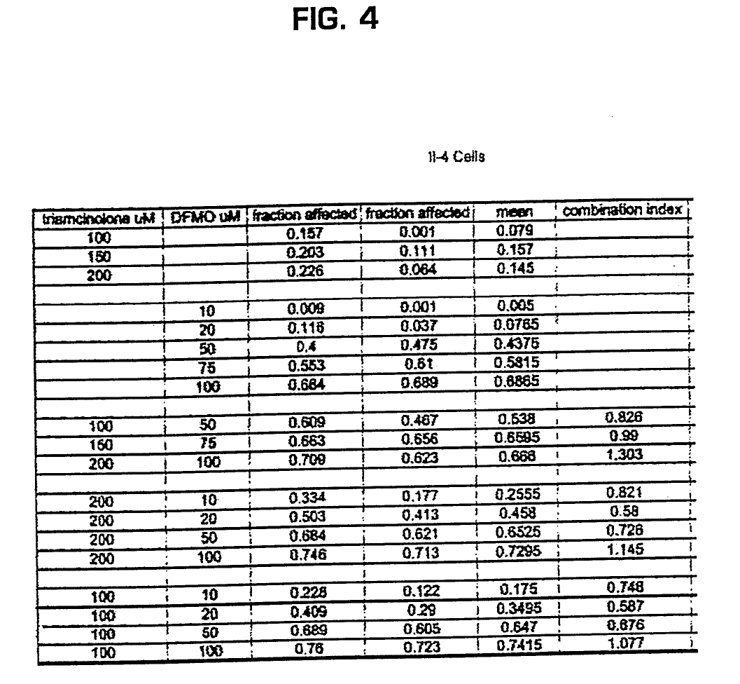

Likewise, the graph set forth in FIG. 3 shows the percent survival rate of Type II-4 squamous cancer skin cells as a function of the microMolar concentration of the alpha-DFMO. The FIG. 3 graph demonstrates that, for concentrations of alpha-DFMO in the range of approximately 10 to 100 microMolar, the addition of 200 microMolar triamcinolone significantly improved the effectiveness of alpha-DFMO alone relative to the reduction of Type II-4 squamous cancer skin cells. All of the data recorded for Type II-4 squamous cancer skin cells is set forth in the data table of FIG. 4.

In a second study, in vivo tests were made using laboratory mice, along with the II-4 human squamous skin cell line. Human keratinocytes were modified for immortalization by transfection of the c-Harvey-Ras (EJ) oncogene, pursuant to the methods described in Boukamp, et al., “c-Ha-ras Oncogene expression in immortalized human keratinocytes (HaCaT) Alters growth potential in vivo but lacks correlation with malignancy”, Cancer Research, 50:2840-2847, 1990, the contents of which are hereby incorporated by reference. This skin squamous cancer cell line, termed HaCaT II-4, has been shown to produce malignant tumor growth when injected subcutaneously into immunologically deficient (SCID) mice. The tumors that form are invasive, but still form an epidermis-like stratified epithelium when transplanted.

In this second study, the topical formulation that was tested consisted of a 10% (w/w) of alpha-DFMO added to 0.1% (w/w) triamcinolone acetonide, along with a topical vehicle. The 10% alpha-DFMO was originally obtained from Marion Merrell, Dow Corp., which later merged into Hoecsht Pharmaceuticals, which licensed all manufacturing and intellectual property rights to ILEX Oncology, San Antonio, Tex. The 0.1% triamcinolone was obtained from Sigma Chemical of St. Louis, Mo. The topical vehicle selected was a Hydrophilic Ointment, USP, obtained from E. Fougera & Co., of Melville, N.Y., although Vanicream® topical vanishing cream or like vehicles can also be used. The powdered drugs were mixed in a blender into the ointment base and stored for later use at room temperature.

The mice used for the second study were severe-combined immune deficiency (SCID) mice provided from the Arizona Cancer Center breeding facility. Sixteen of such mice were divided into four groups in the manner explained below. The tumor cells were initially grown in vitro in sterile cell culture in sufficient quantity to inject each mouse with 10 million cells; these cells were injected subcutaneously into the front flank muscle on Day 0 of the study. The relative time at which topical treatment was started was varied among three groups of mice; one group of mice received pre-treatment starting ten days before tumor implantation, a second group of mice began receiving topical treatment one day after tumor implantation (before any tumor was palpable), and a third group of mice began receiving treatment eight days after tumor implantation (when a mean 30 mm³ tumor was palpable). A fourth group of mice served as a control group, and were treated with pure hydrophilic ointment, lacking any alpha-DFMO or triamcinolone, beginning the first day after tumor implantation. Topical treatment consisted of the application of 100 μL of the above-described ointment mixture delivered by positive-displacement pipette daily. The control group received an equal amount of pure hydrophilic ointment.

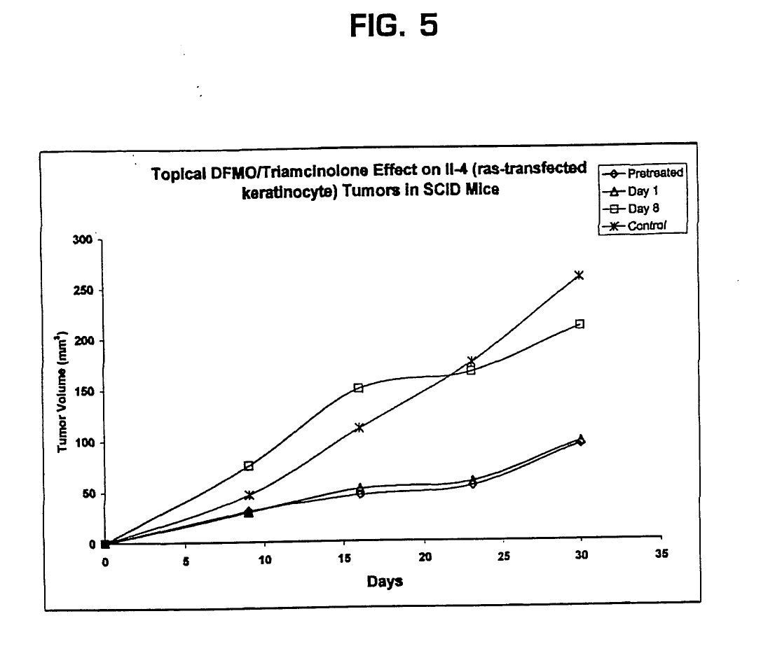

The results of this second study are depicted within the graph of FIG. 5. Significant anti-tumor activity (reduced rate of tumor growth) was observed in both the first (pre-treatment) and second (treatment beginning on Day 1) groups of mice as compared with the control group. However, the third group of mice (treatment delayed until Day 8) did not demonstrate significant tumor growth inhibition as compared with the control group. Within this second study, there was no visual evidence of local toxicity at the application site of the topical treatment at any time during the study.

This second study indicated that the combination of topical alpha-DFMO plus triamcinolone was tolerated when applied topically to SCID mice. The results of the second study further demonstrate that when the combination of topical alpha-DFMO plus triamcinolone is applied topically in appropriate amounts, prior to the development of a palpable tumor mass, such treatment inhibits growth of a human keratinocyte-derived squamous cell cancer.

In a third study of the effects of combining alpha-DFMO and triamcinolone, SCID mice (4/group) were given 10⁷ II-4 cells and then randomized to receive topical treatment with triamcinolone acetonide (0.05% or 0.1%), α-DFMO, (5% or 10%), or the combination of α-DFMO with triamcinolone. The control groups received either no treatment (n=4), or the ointment base (hydrophilic ointment, U.S.P. alone (n=4), or the ointment base (hydrophilic ointment, U.S.P. alone (n=4). Treatments were given daily to the tumor-bearing blank skin continuously beginning 24 hours after tumor implantation. Palpable tumors were measured bi-dimensionally using calipers, three times per week, until tumor sizes approximated 1,500 mm³ (about 1.5 g). The mice were then euthanized.

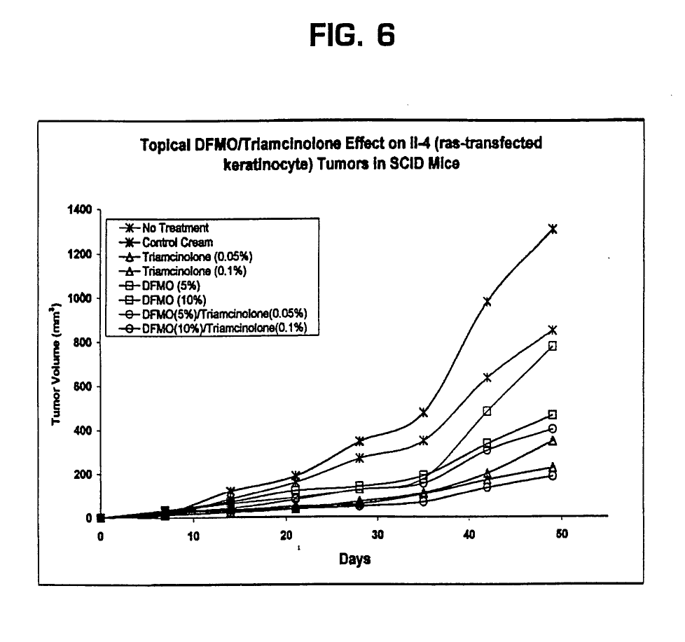

The results of this third study are shown in the graph of FIG. 6. In the graph of FIG. 6, tumor cell volume is plotted as a function of time over a period of fifty days for no treatment; hydrophilic ointment only; 5% and 10%, respectively, alpha-DFMO only; 0.05% and 0.1%, respectively, of triamcinolone only; and the combinations of 5% alpha-DFMO/0.05% triamcinolone, and 10% alpha-DFMO/0.1% triamcinolone. The graph of FIG. 6 shows that tumor cell volume grew the fastest either when there was no treatment, or when the topical application consisted merely of hydrophilic ointment. The rate of growth for hydrophilic ointment alone is actually faster than for no treatment at all, indicating that the rubbing of such tumor cells through topical application of the ointment actually stimulates tumor growth. The graph of FIG. 6 further illustrates that the application of alpha-DFMO by itself decreases the rate of tumor growth, and that the application of triamcinolone by itself decreases the rate of tumor growth. Finally, the graph of FIG. 6 demonstrates that the tumor growth rate was reduced the most by the combination of 10% (by weight) alpha-DFMO and 0.1% triamcinolone.

Thus, topical application of alpha-DFMO plus triamcinolone not only inhibits potentially-cancerous actinic keratoses cells from becoming cancerous (chemo-prevention of tumor development), but actually reduces the number of cells that have already become cancerous (inhibition of frank tumor growth). Thus, the combination is believed to be effective in treating pre-cancerous conditions, including lesions and non-invasive carcinoma insitu of cutaneous and muco-cutaneous regions including actinic keratoses, vulvar neoplasia, and pre-cancerous conditions of the lip, nostrils, nails, and anus. Where pre-cancerous lesions are treated, the combination may be effective in downgrading the lesions (i.e. from VIN III to VIN II or VIN I, in the case of vulvar neoplasia).

Relative concentrations of topical alpha-DFMO believed to be effective lie within the range of 0.1% to 20% by weight. Likewise, relative concentrations of topical triamcinolone which are believed to be effective lie within the range of 0.001% to 1.0% by weight. Preferred relative ratios by weight of alpha-DFMO to triamcinolone combined with the base vehicle ranges between 0.10:2 and 40:1. The alpha-DFMO and triamcinolone may be formulated as a single composition or as two separate compositions which could be applied separately at different times of the day. For example, for vulvar application, the DFMO cream may be applied daily in the morning immediately after a bath or shower; a two-inch strip of 10% alpha-DFMO cream is applied to the entire vulva. The triamcinolone cream may be applied each night at bedtime to the entire vulva. Additionally, the two separate compositions may be stored separately, and then mixed prior to application.

The applicants conducted a further study to investigate the effects of combining topical alpha-DFMO with topical diclofenac (a topical non-steroid anti-inflammatory) as a method for treating actinic keratoses by topical application to the skin tissues of a human being containing actinic keratoses lesions. In this study, the effect of such combination was investigated in regard to II-4 squamous cancer cells. In preparing the topical application for the study, the same hydrophilic ointment mentioned above was used as a base vehicle. Alpha-DFMO in the amount of 5% (w/w) by weight was added to the base vehicle. In addition, 1% (w/w) by weight diclofenac was combined therewith. The resulting combination was applied to SCID mice given 10⁷ II-4 cells in the flank one day previously.

Within the article by Alberts, et al., “Pharmacologic Studies of Anticancer Drugs with the Human Tumor Stem Cell Array”, Cancer Chemother Pharmacol, 1981, 6:253-264, the contents of which are hereby incorporated by reference, an explanation is provided for assessing the treatment efficacy of combining two or more drugs. The method described in such article is based upon the so-called “Fractional Survival Method of Drewinko, et al.”. This article describes the difference between combinations which are merely additive, combinations which are antagonistic, and combinations that render truly synergistic results.

The test results obtained from the above-described combination of topical alpha-DFMO with topical diclofenac indicated that the experimentally-obtained fractional survival effect of such combination was significantly improved over the fractional survival effect that would have been predicted algebraically by simply multiplying together the individual fractional survival effects of alpha-DFMO and diclofenac when used alone. Thus, the combination is believed to be effective in treating pre-cancerous conditions, including lesions and non-invasive carcinoma insitu of cutaneous and muco-cutaneous regions including actinic keratoses, vulvar neoplasia, and pre-cancerous conditions of the lip, nostrils, nails, and anus. Where pre-cancerous lesions are treated, the combination may be effective in downgrading the lesions (i.e. from VIN III to VIN II or VIN I, in the case of vulvar neoplasia). The preferred concentration of topical diclofenac is within the range of 0.1%-10% by weight, while the alpha-DFMO has a preferred concentration within the range of 0.10% to 20% by weight. The relative ratio by weight of alpha-DFMO to topical diclofenac combined with the base vehicle preferably ranges between 2:1 and 20:1

While the aforementioned study used diclofenac as the topical non-steroid anti-inflammatory, other suitable non-steroidal anti-inflammatory compounds include ketoprofen, ibuprofen, celecoxib, salicylate, difinisal, etodolac, fenoprofen, ketorolac, mefenamic acid, nabumetone, naproxen, oxaprozin, tolmetin sodium, and rofecoxib.

A further in vitro study was conducted by the applicants to investigate the efficacy of the topical application of alpha-DFMO, triamcinolone, and diclofenac as a method for treating actinic keratoses by topical application to the skin tissues of a human being. Alpha-DFMO and triamcinolone were added together with diclofenac. This study was directed to II-4 human squamous cell skin cancer cells.

The cell culture was maintained as a monolayer culture. The cells were plated in 96 well microtiter plates on Day 0 of the study. Some of such wells were used as control wells, so no drugs were added thereto. The aforementioned combination of drugs was added to the microtiter test wells on Day 1. On Day 7, the plates were fixed with cold 10% trichloroacetic acid, and then washed four times with distilled water. Each plate was then stained with 0.4% sulforhodamine B. Excess stain was then removed by washing the plate four times with 1% acetic acid. The stained dye is then solubilized with 50 mM Tris. The stained plates were then “read”, i.e., measured for optical density, on an automatic plate reader at 540 nm. A lower optical density corresponds to a smaller volume of cancerous cells, and a higher optical density corresponds to a larger volume of cancerous cells. A surviving fraction factor fₛ is then computed for the treated well plates by taking the mean optical density of the treated well plates and dividing by the mean optical density of the control well plates.

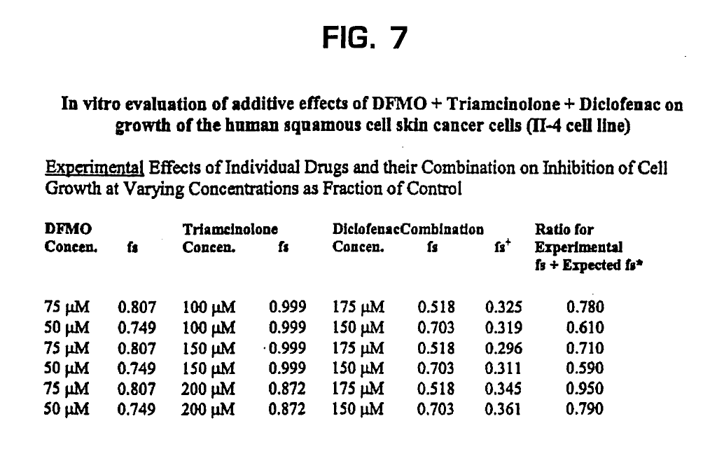

The results of this in vitro study on the three-drug combination are set forth in the table of FIG. 7. Within the first two columns of FIG. 7, the individual surviving fraction factors fₛ (0.749 and 0.807) are set forth for two different concentrations (50 μM and 75 μM, respectively) of alpha-DFMO. While a lower surviving fraction factor 0.749 is associated with the smaller dosage of 50 μM, and a higher surviving fraction factor 0.807 is associated with the greater dosage of 75 μM, the difference in such numbers is probably not statistically significant. Likewise, in the second pair of columns of the table of FIG. 7, the individual surviving fraction factors fₛ (0.999, 0.999 and 0.872) are set forth for three different concentrations (100 μM, 150 μM, and 200 μM, respectively) of triamcinolone. It has been noted that the surviving fraction factor 0.999 is the same for both 100 μM and 150 μM, whereas increasing the dosage from 150 μM to 200 μM produces a noticeable improvement; this could be due to a threshold effect that is overcome at doses exceeding 150 μM. The third pair of columns in the table of FIG. 7 show the individual surviving fraction factors fₛ (0.703 and 0.518) for two different concentrations (150 μM and 175 μM, respectively) of diclofenac. The column entitled fₛ+ shows the actual surviving fraction factor measured in the laboratory for each such three-drug combination.

As explained in the aforementioned article by Alberts, et al., a mathematically-derived expected fraction factor (fₛ*) can be computed for each three-drug combination by multiplying together the individual fraction factors for the three individual drugs. A ratio can then be computed for the actual experimentally-determined fraction factor fₛ divided by the expected fraction factor fₛ*, and this ratio is presented in the rightmost column of the table in FIG. 7. As further explained in the article by Alberts, et al., a ratio of 1:1 indicates that a drug combination is additive, whereas a ratio of less than 1:1 indicates that the drug combination is synergistic. The lower the ratio, the more synergistic is the combination. As will be noted, all of the ratio values in the rightmost column of the table of FIG. 7 are less than 1:1, with at least two of the combinations producing ratios as low as approximately 0.6:1. Thus, the combination of alpha-DFMO, triamcinolone, and diclofenac is believed to be effective in topically treating pre-cancerous conditions, including lesions and non-invasive carcinoma insitu of cutaneous and muco-cutaneous regions including actinic keratoses, vulvar neoplasia, and pre-cancerous conditions of the lip, nostrils, nails, and anus. Where pre-cancerous lesions are treated, the combination may be effective in downgrading the lesions (i.e. from VIN III to VIN II or VIN I, in the case of vulvar neoplasia).

Those skilled in the art will now appreciate that an improved topical combination and method for treating pre-cancerous and cancerous conditions occurring in cutaneous and muco-cutaneous regions has been described which is highly effective in topically treating conditions such as actinic keratoses and vulvar neoplasia, and pre-cancerous conditions occurring in the lip, nails, nostrils, and anus, in humans. The disclosed combination and method is less toxic than known treatment methods, and lessens the likelihood of rashes when alpha-DFMO is used by itself. Indeed, in past studies, when alpha-DFMO was used alone without a topical steroid or non-steroidal anti-inflammatory, to treat actinic keratoses, there was approximately a 20% incidence of skin reactions; in contrast, studies conducted to date for the combination of alpha-DFMO plus triamcinolone show no topical hypersensitivity reactions. The new combination also provides a new method for treating vulvar neoplasia which is much more likely to be tolerated than currently available methods. Such treatment is expected to be effective on lesions occurring in the epithelium as well as deeper layers such as the dermis. The disclosed method unexpectedly enhances the effectiveness of both alpha-DFMO and diclofenac when topically treating pre-cancerous conditions in humans. Perhaps most importantly, the compositions and methods of the present invention are effective not only to prevent pre-malignant conditions from progressing to malignant squamous cell cancer, but may also be effective in inhibiting and/or reducing the growth rate of existing squamous cell cancer. Furthermore, the combination of the present invention is believed to be effective in treating pre-cancer of the mouth or oral triplacia. Though topical application to the inside of the mouth may be more difficult, it is preferably achieved by providing a thick gel or cream, and applying to the affected area, preferably during the middle of the night when the mouth is dry.

While the present invention has been described with respect to preferred embodiments thereof, such description is for illustrative purposes only, and is not to be construed as limiting the scope of the invention. Though, preferably, the compositions of the present invention are combined with a base vehicle such as a hydrophilic ointment (Hydrophilic Ointment, USP), Vanicream® topical vanishing-cream, water-in-oil emulsions and oil-in-water emulsions, it should be understood that the compositions may be present in any known form, including any type of cream, lotion, gel, serum (such serum may be encapsulated or topically administered using a dropper or pipette, for example), etc.

The generic name for alpha-DFMO is eflomithine, which is a racemic mixture of two enantiomers. It has been theorized that one of such enantiomers may be associated with a slight risk of ototoxicity (hearing loss), though Applicants do not believe that any systemic toxicity results from the topical application of racemic topical alpha-DFMO because there was negligible systemic uptake in animal studies, and no drug was detected in clinical trials following topical application of alpha-DFMO. Nonetheless, it may be possible to obtain the advantages of the present invention as described above using only one or the other of the two enantiomers that form the racemic mixture of alpha-DFMO; accordingly, the use of the term “alpha-DFMO” herein should be understood, for purposes of the present patent application, to include both the racemic mixture of the two enantiomers that normally make up alpha-DFMO, as well as each of the two enantiomers that collectively make up the racemic mixture of alpha-DFMO.

Various modifications and changes may be made to the described embodiments by those skilled in the art without departing from the true spirit and scope of the invention as defined by the appended claims.

We claim:

1. A method for treating a pre-cancerous conditions in an affected cutaneous or muco-cutaneous region of a human being, comprising the steps of:
topically applying alpha-DFMO to said affected region; and
topically applying a steroidal anti-inflammatory agent to said affected region.

2. The method of claim 1 wherein said condition is actinic keratoses, vulvar, or pre-cancerous cells present in the vulva, lip, nostrils, nails, or anus.

3. The method of claim 1 wherein said pre-cancerous condition includes pre-cancerous lesions, non-invasive carcinoma insitu, or a combination thereof.

4. The method of claim 1 wherein said steroidal anti-inflammatory agent has a relative concentration within the range of 0.001% to 1.0% by weight.

5. The method of claim 1 wherein said steroidal anti-inflammatory agent has a concentration within the range of 0.01 to 1.0% by weight.

6. The method of claim 1 wherein the alpha-DFMO has a concentration within the range of 0.1% to 20% by weight.

7. The method of claim 1 wherein the relative ratio by weight of alpha-DFMO to said steroidal anti-inflammatory agent ranges between 0.10:2 and 40:1.

8. The method of claim 1, further comprising combining the alpha DFMO and steroidal anti-inflammatory agent with at least one base vehicle for topical application.

9. The method of claim 8 wherein the base vehicle is Vanicream® topical vanishing cream, a hydrophilic ointment, a water-in-oil emulsion, an oil-in-water emulsion, a gel, a serum, or a combination thereof

10. The method of claim 8 wherein the alpha-DFMO and steroidal anti-inflammatory agent are combined in one base vehicle.

11. The method of claim 8 wherein the alpha-DFMO is combined with a first base vehicle, and the steroidal anti-inflammatory agent is combined with a second base vehicle to form first and second compositions, said method further comprising applying said first composition and applying said second composition to said region, wherein the time interval between applications ranges from 0-24 hours.

12. The method of claim 8 wherein the alpha-DFMO is combined with a first base vehicle, and the steroidal anti-inflammatory agent is combined with a second base vehicle to form first and second compositions, said method further comprising mixing said first and second compositions to form a mixture prior to application, and applying said mixture.

13. The method of claim 1 wherein said steroidal anti-inflammatory agent is selected from the group of topical steroids consisting of triamcinolone, betamethasone, clobetasol, dexamethasone, furoate, fluocinonide, amcinonide, desonide, desoximetasone, fluocinolone, fluticasone, halobetasol, hydrocortisone, and mometasone.

14. The method of claim 1, further comprising topically applying a non-steroidal anti-inflammatory agent to said affected region.

15. The method of claim 14 wherein the topical non-steroid anti-inflammatory agent is selected from the group of non-steroidal anti-inflammatory compounds consisting of diclofenac, difunisal, etodolac, fenoprofen, ketoprofen, ketorolac, mefenamic acid, nabumetone, naproxen, oxaprozin, tolmetin sodium, ibuprofen, celecoxib, rofecoxib, choline salicylate and sodium salicylate.

16. The method of claim 14 wherein the topical steroidal agent is triamcinolone, and the non-steroidal agent is diclofenac.

17. The method of claim 14, further comprising combining the alpha DFMO, the steroidal agent, and the non-steroidal agent with at least one base vehicle for topical application, said base vehicle comprising Vanicream® topical vanishing cream, a hydrophilic ointment, a water-in-oil emulsion, an oil-in-water emulsion, a gel, a serum, or a combination thereof.

18. The method of claim 17 wherein said non-steroidal agent is formulated separately then mixed with the alpha-DFMO, steroidal agent, or both, prior to application.

19. The method of claim 17 wherein said non-steroidal agent is formulated and applied separately.

20. The method of claim 17 wherein said alpha-DFMO, steroidal agent, and non-steroidal agent are together combined with said base vehicle.

21. A method for treating a pre-cancerous condition in a region of the body of a human being affected with the condition, the method comprising the steps of:
topically applying alpha-DFMO to said region affected with said pre-cancerous condition; and
topically applying an anti-inflammatory agent selected from the group comprising a steroidal anti-inflammatory agent, a non-steroidal anti-inflammatory agent, or a combination thereof, said region of the body selected from the group consisting of the vulva, lip, mouth, nails, nostrils, and anus.

22. A method for treating a pre-cancerous condition in a region the body of a human being affected with the condition, the method comprising the steps of:
topically applying a first composition containing alpha-DFMO to said region affected with said pre-cancerous condition; and
topically applying a second composition, separate from said first composition, comprising an anti-inflammatory agent selected from the group comprising a steroidal anti-inflammatory agent, a non-steroidal anti-inflammatory agent, or a combination thereof, said region of the body selected from the group consisting of the skin, vulva, lip, mouth, nails, nostrils, and anus.

23. The method recited by claim 22 wherein said second composition comprises diclofenac.
